(19) United States
(12) Patent Application Publication
(10) Pub. No.: US 2026/0087622 A1
(43) Pub. Date: Mar. 26, 2026

(54) METHOD AND SYSTEM FOR OPTIMAL DETERMINATION OF VESSEL BRANCHES FOR TARGET COVERAGE WITH MINIMUM EXPOSURE TO HEALTHY TISSUES

(71) Applicant: EDDA TECHNOLOGY, INC., PRINCETON, NJ (US)

(72) Inventors: Xin Dou, Princeton, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 19/331,625

(22) Filed: Sep. 17, 2025

Related U.S. Application Data
(60) Provisional application No. 63/698,726, filed on Sep. 25, 2024.

(51) Int. Cl. G06T 7/00; A61N 5/10; G06T 7/73; G06V 10/25; G16H 50/50

(52) U.S. Cl. CPC G06T 7/0012; G06T 7/73; G06V 10/25; G16H 50/50; A61N 5/1007; A61N 2005/1012; G06T 2200/04; G06T 2207/30096; G06T 2207/30101; G06V 2201/03

(57) ABSTRACT

The present teaching relates to determination of an optimal set of supplying vessel branches for delivering radioactive material to a target region. A centerline representation for blood vessels is obtained based on a 3D image capturing an organ with a target region and a non-target region. Supplying centerline segments, each representing a supplying vessel branch, are identified based on connected supplying centerline points that cover some parts of the target region. An optimal set of supplying vessel branches are selected that satisfy a specified coverage to the target region with a least coverage to the non-target region.

### CROSS REFERENCE TO RELATED APPLICATION

The present application claims the benefit and priority of U.S. Provisional Patent Application No. 63/698,726, entitled “METHOD AND SYSTEM FOR OPTIMAL DETERMINATION OF VESSEL BRANCHES FOR TARGET COVERAGE WITH MINIMUM EXPOSURE TO HEALTHY TISSUES”, filed on Sep. 25, 2024, the contents of which are hereby incorporated by reference in its entirety.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including the medical industry, where large volume of image data, capturing anatomical information of a patient, may be processed by computers to identify anatomical structures of interest (e.g., organs, bones, blood vessels, or abnormal nodule), obtain measurements thereof (e.g., dimension of a nodule growing in an organ), and leverage such information in different applications. For example, a 3D model may be constructed for an organ (e.g., a liver in terms of its volume, shape, or size along different dimensions), different organic parts nearby the organ (e.g., a nodule growing inside a liver, vessel structures inside and near the organ and or the nodule). Such information may be used in, e.g., tumor treatment through vascular interventions.

Some medical procedures for treating abnormal tumor involve vascular interventions, such as in tumor resection or selective internal radiation therapy. In the former procedure, vessels supplying blood to a tumor may be clamped to reduce the blood flow and enhance the visibility. In the selective internal radiation therapy, vessels supplying blood to a tumor is used to inject microspheres containing radioactive material to deliver radiation directly to the tumor cells. FIG. 1A illustrates an example, where an organ 100 includes a sub-structure therein having two parts 110 and 120. An abnormal tumor 130 is located within the organ 100, occupying a sub-regions in both parts 110 and 120. In this example, a blood vessel supplies blood to the tumor 130 in both parts 110 and 120 as well as healthy tissues of the sub-structure via branches 140, 150, and 160. In this example, while blood vessels 150 and 160 are identified as blood supplying vessels for tumor 130, they also supply blood to other healthy tissues in the organ 100. As illustrated in FIG. 1B, if microspheres with radioactive material is injected at an injection point 180 on vessel 160, the radioactive material reaches an area 190, that is partly within the tumor 130 and partly outside the tumor with healthy tissues. The goal in such a therapy is to deliver the radiation directly to the tumor (as complete as possible) while sparing as much surrounding healthy tissue as possible.

Such vascular intervention procedures target the blood supply sources (vessels) to ensure certain effect on an intended area (such as a tumor). Thus, it is crucially important to accurately locate relevant blood supplying vessel branches with respect to a targeted area (tumor). Traditional methods for locating relevant vessels are generally based on anatomical landmarks or manual assessment, which can be imprecise, yielding variable performance. In some situations, such methods for localizing blood-supplying arteries rely on statis images which may not capture important information in the dynamic situation during a procedure such as the dynamics of the blood flow and individual anatomical variations of the patient while on a surgical table. This can lead to suboptimal execution, affecting the treatment negatively. Thus, there is a need for a solution that addresses the challenges discussed above.

### SUMMARY

The teachings disclosed herein relate to methods, systems, and programming for information management. More particularly, the present teaching relates to methods, systems, and programming related to content summarization.

In one example, a method is disclosed for determination of an optimal set of supplying vessel branches for delivering radioactive material to a target region. A centerline representation for blood vessels is obtained based on a 3D image capturing an organ with a target region and a non-target region. Supplying centerline segments, each representing a supplying vessel branch, are identified based on connected supplying centerline points that cover some parts of the target region. An optimal set of supplying vessel branches are selected that satisfy a specified coverage to the target region with the least coverage to the non-target region.

In a different example, a system is disclosed for determination of an optimal set of supplying vessel branches for delivering radioactive material to a target region. The system is disclosed to include a model-based vessel centerline constructor and a supplying vessel branch determiner. The model-based vessel centerline constructor is provided for generating a centerline representation with a plurality of centerline points representing the one or more blood vessels based on a three-dimensional (3D) image with voxels capturing an organ with target and non-target regions and one or more blood vessels therein. The supplying vessel branch determiner is for identifying, from the centerline representation, at least one supplying centerline segment, each of which includes multiple connected supplying centerline points and represents a supplying vessel among the blood vessels, and selecting an optimal set of supplying vessels that satisfy a specified coverage to the target region with a least coverage to the non-target region.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory, and tangible medium having information recorded thereon for determination of an optimal set of supplying vessel branches for delivering radioactive material to a target region. When the information is read by the machine, it causes the machine to perform various steps. A centerline representation for blood vessels is obtained based on a 3D image capturing an organ with a target region and a non-target region. Supplying centerline segments, each representing a supplying vessel branch, are identified based on connected supplying centerline points that cover some parts of the target region. An optimal set of supplying vessel branches are selected that satisfy a specified coverage to the target region with the least coverage to the non-target region.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities, and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIGS. 1A-1B show example spatial relations among an organ, a tumor, and blood supplying vessels and medical effect thereof when a vessel is injected with microsphere having radioactive material;

FIGS. 2A-2C depict an exemplary blood vessel tree with branches and automatically determined blood supplying vessel branches with injection points for injecting microsphere during a treatment, in accordance with an embodiment of the present teaching;

FIG. 3A depicts an exemplary system diagram of a framework for automatically determining blood supplying vessel branches and injection points thereon with respect to a target region and a non-target region, in accordance with an embodiment of the present teaching;

FIG. 3B is a flowchart of an exemplary process for a framework for automatically determining blood supplying vessel branches and injection points thereon with respect to a target region and a non-target region, in accordance with different embodiments of the present teaching;

FIG. 3C illustrates an exemplary centerline representation of a blood vessel tree;

FIG. 4A depicts an exemplary system diagram of a supply vessel branch determiner, in accordance with an embodiment of the present teaching;

FIG. 4B illustrates examples of identifying a vessel centerline point that supplies blood to a point in a region, in accordance with an embodiment of the present teaching;

FIG. 4C illustrates an example of statistics recorded with respect to each vessel centerline point, in accordance with an embodiment of the present teaching;

FIG. 5A is a flowchart of an exemplary process of a supply vessel branch determiner to identify blood supplying centerline points, in accordance with an embodiment of the present teaching;

FIG. 5B is a flowchart of an exemplary process of a supply vessel branch determiner to generate an optimal set of blood supplying branches with respect to a target region, in accordance with an embodiment of the present teaching;

FIG. 6A shows an example centerline representation of a vessel tree with some centerline points identified as blood supplying points, in accordance with an embodiment of the present teaching;

FIG. 6B shows an example of filtering operation performed on blood supplying points on a vessel centerline representation, in accordance with an embodiment of the present teaching;

FIG. 6C shows an example of forming centerline representations of blood supplying vessel branches identified with respect to a target region, in accordance with an embodiment of the present teaching;

FIG. 6D shows an example of separate blood supplying vessel branches with common parent node, in accordance with an embodiment of the present teaching;

FIG. 6E shows an example of merging two separate blood supplying vessel branches with common parent node to create a merged blood supplying vessel branch, in accordance with an embodiment of the present teaching;

FIG. 6F illustrates an optimization result where some supplying blood vessel branches are automatically identified and selected to achieve maximal coverage of a target region with minimized reach to non-target region, in accordance with an embodiment of the present teaching;

FIG. 7 is an illustrative diagram of an exemplary mobile device architecture that may be used to realize a specialized system implementing the present teaching in accordance with various embodiments; and

FIG. 8 is an illustrative diagram of an exemplary computing device architecture that may be used to realize a specialized system implementing the present teaching in accordance with various embodiments.

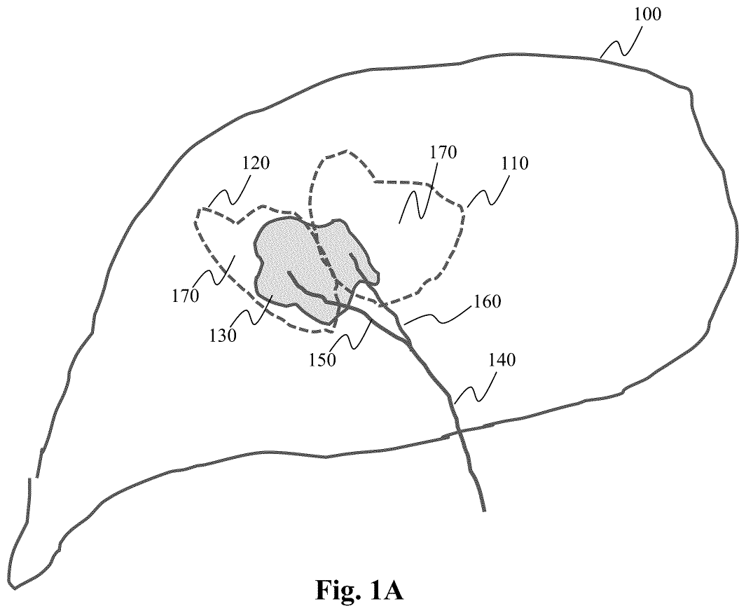

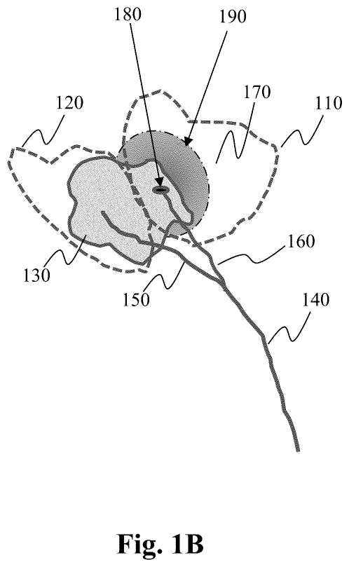

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

The present teaching discloses exemplary methods, systems, and implementations for automatically identifying blood supplying vessel branches for injecting microsphere with radioactive material to a target region in an organ. The purpose of determining an optimal set of blood supplying vessel branches is to achieve a maximum coverage of the target region such as a tumor and a minimum damage to healthy tissues in a non-target region such as tissues around a tumor. Blood vessels associated with a target region in an organ may be identified. In some embodiments, such relevant blood vessels may be identified based on 3D models constructed for the organ, which may include a 3D model for the target area (tumor), 3D models for blood vessels connected or around the target area, and optionally a 3D model for the organ. The identified blood vessels may be relied on to obtain a corresponding centerline representation, which may then be used to determine supplying blood vessels for delivering radioactive material to the target region while minimizing the damage to surrounding non-target regions.

To identify supplying blood vessel branches, with respect each point (e.g., a 3D voxel in a 3D image) in the organ (either in a target region or in a non-target region), a corresponding point on the centerline representation of the blood vessels may be determined as a supplying centerline point. In some embodiments, a corresponding centerline point may be one that is closest to the point in the organ. A centerline point may be assessed as to whether it is a supplying centerline point based on two statistics, one being a total number of target points in the organ closest thereto and the other being a total number of non-target points the centerline point is closest to. If the number of target point that a centerline point is the closest thereto, then the centerline point may be deemed as a supplying centerline point, and it covers the recorded number of target points in the organ.

Adjacent supplying centerline points may be connected to form a centerline representation of a supplying blood vessel branch. The coverage of a supplying vessel branch may be the sum of the numbers of target points covered by the connected supplying centerline points. In some embodiments, the number of non-target points of the supplying blood vessel branch may be the sum of the numbers of non-target points of the connected supplying centerline points, which correspond to the damage to healthy tissues when the supplying blood vessel branch is used for injecting radioactive material. In some embodiments, two supplying blood vessel branches may be merged if their centerline representations may trace to a common parent centerline point. When two supplying blood vessel branches are merged, the coverage of both target and non-target regions of the merged branch may be the summations of the corresponding coverages of the two component branches.

To optimize the selection of supplying blood vessel branches to ensure full or maximum coverage of the target region while minimize the damages to non-target region, some supplying blood vessel branches may be determined that can reach a balance of a maximum coverage of target region while minimizing the affected non-target region. In some embodiments, based on the optimized set of supplying blood vessel branches, an appropriate point on each of the supplying blood vessel branches may be determined as the injection point to deliver, via this supplying blood vessel branch, radioactive material to the target region. In some embodiments, some criterion may be utilized to determine the injection point. For example, an injection point may not be close to a fork point.

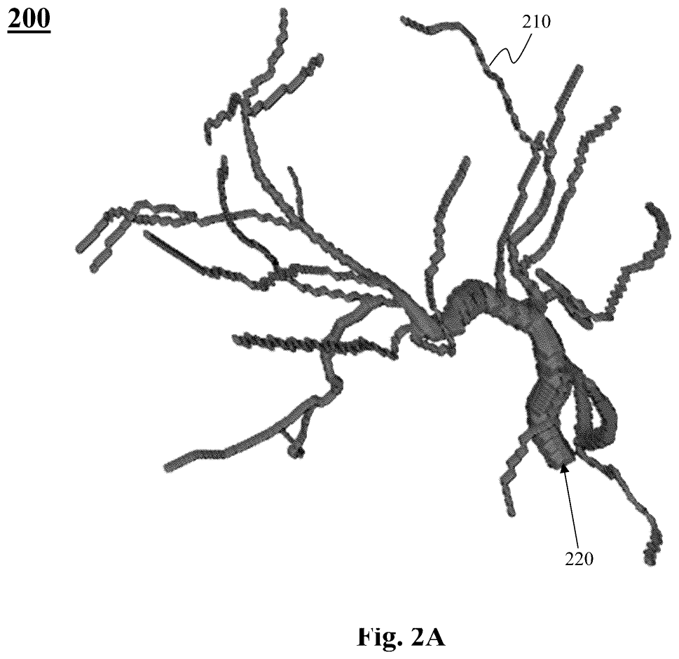

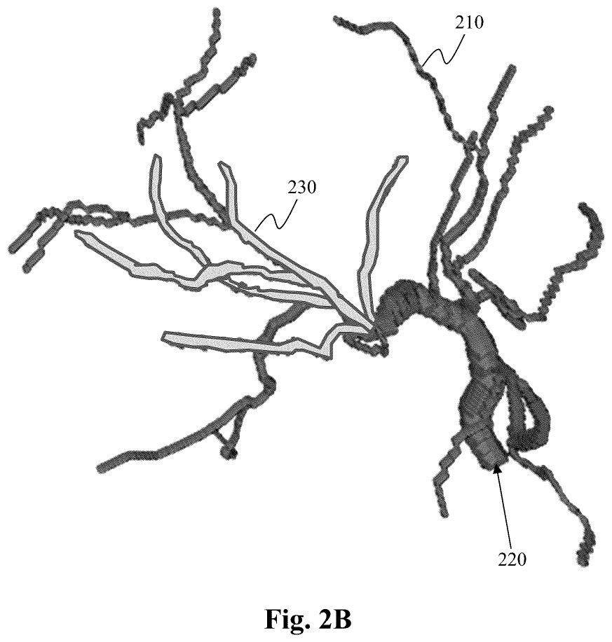

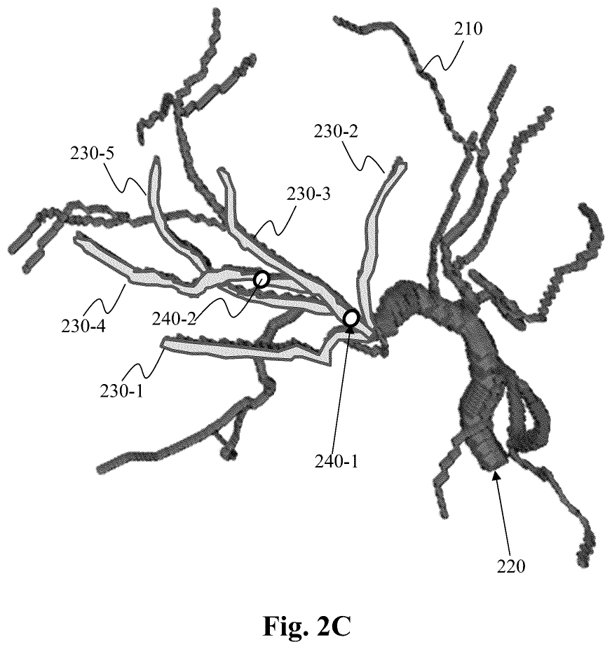

FIGS. 2A-2C depict an example of supplying blood vessel branches identified from a blood vessel tree with respect to a target region according to an embodiment of the present teaching. FIG. 2A shows an exemplary blood vessel tree 200 with branches such as 210 and a root of the tree 220. In this example, a vessel tree 200 includes a root 220 and various branches. Based on the spatial relations between different vessel branches and a target region (not shown), the goal is to select some of the vessel branches as delivery paths to inject radioactive material to the target region. FIG. 2B illustrates an example vessel branches 230 and FIG. 2C shows specific vessel branches 230-1,230-2, 230-3, 230-4, and 230-5, which are selected to deliver radioactive material to a target region with automatically determined injection point 240, in accordance with an embodiment of the present teaching. In addition, as shown in FIG. 2C, some injection points along the selected supplying blood vessel branches are marked as locations to deliver radioactive material to the target region.

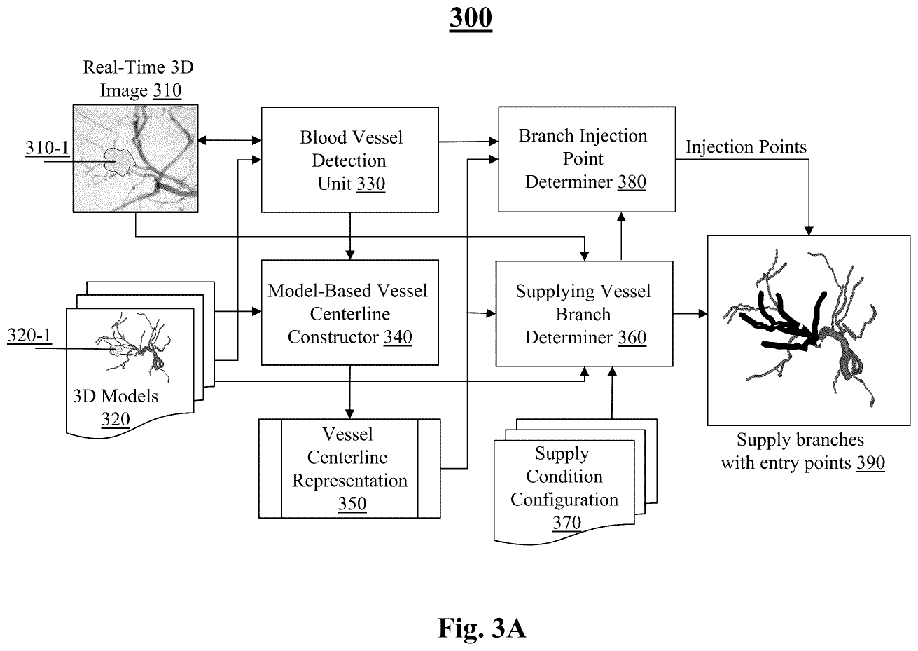

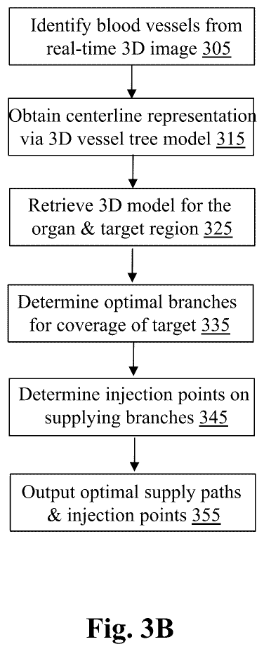

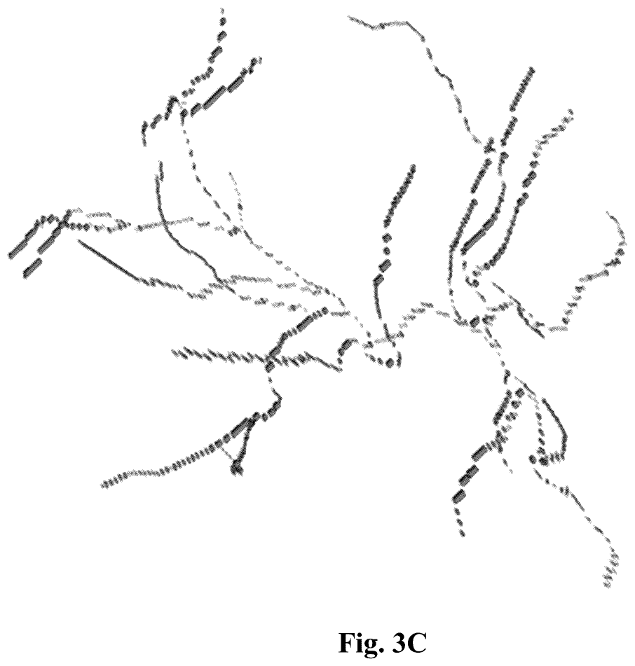

FIG. 3A depicts an exemplary system diagram of a framework 300 for automatically determining supplying blood vessel branches and injection points thereon with respect to a target region and a non-target region, in accordance with an embodiment of the present teaching. In this illustrated embodiment, the framework 300 takes a real-time 3D image 310 and 3D models 320 as input and generate supplying blood vessel branches with injection points 390 as output. The input real time 3D image captures a 3D image with an organ, blood vessels therein, and a target region such as a tumor 310-1. The input 3D models 320 may include a model for an organ, a model 320-1 for the target region or a tumor, and 3D models for various blood vessels within or around the organ. The 3D model for the tumor specifies the target region for delivering radioactive material. The input real time 3D image also captures the tumor during a medical procedure and may be used, in connection with the 3D model for the tumor, to determine which of the vessel branches to be used for injecting radioactive material to the tumor with the goal of achieving a maximum coverage of the tumor (target region) yet with minimized damage to regions outside of the tumor (non-target region).

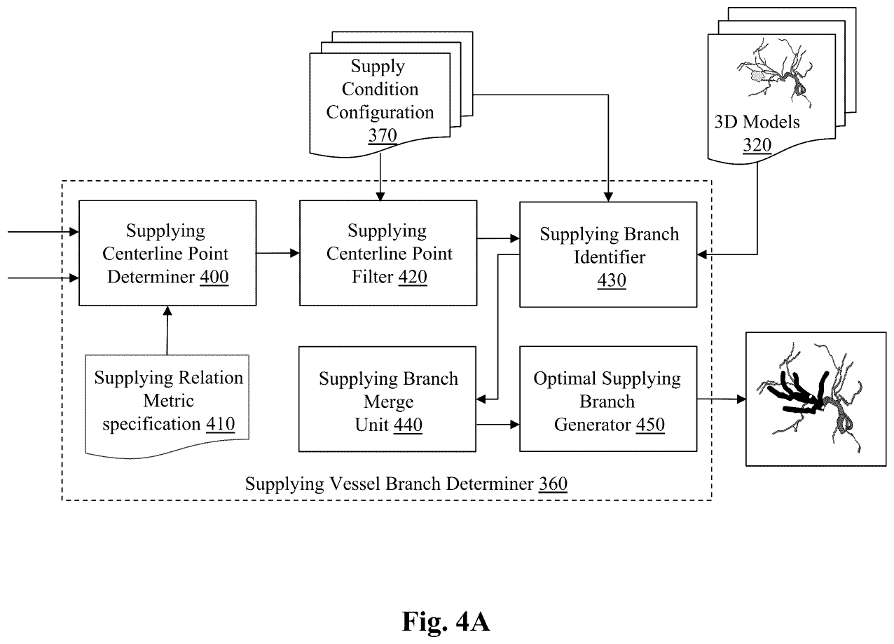

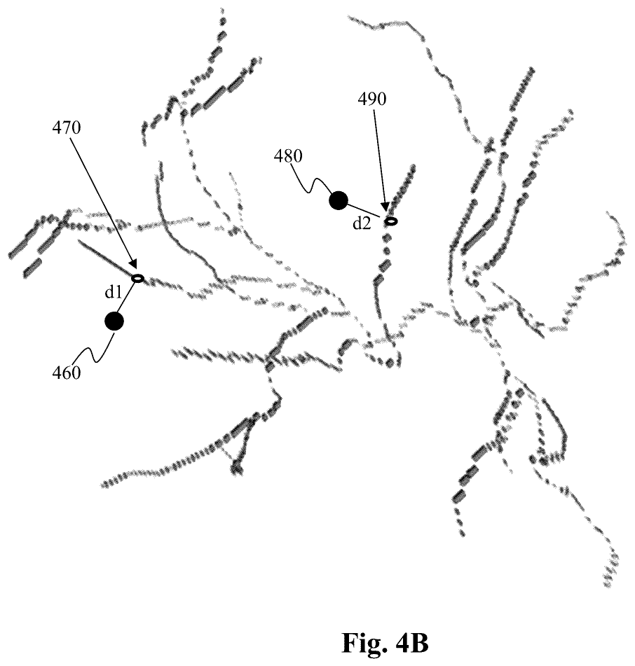

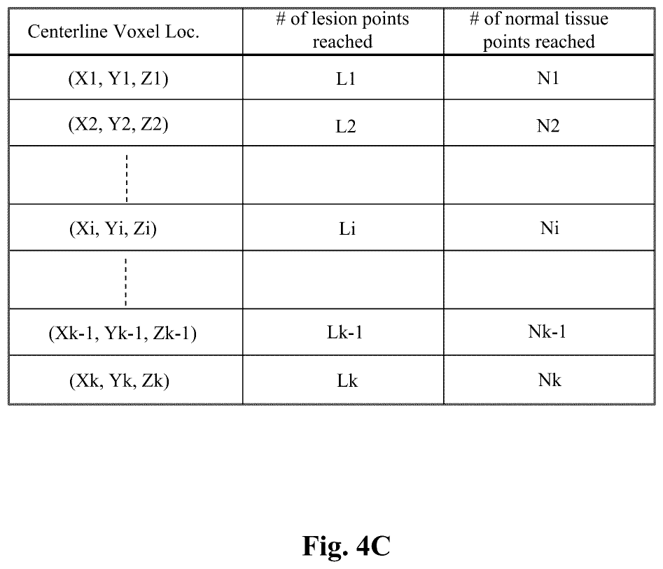

In the illustrated embodiment, the framework 300 comprises a blood vessel detection unit 330, a model-based vessel centerline constructor 340, a supplying vessel branch determiner 360, and a branch injection point determiner 380. The blood vessel detection unit 330 may be provided to detecting, from the input real-time 3D image 310, blood vessel structures. In some embodiment, 3D models for blood vessels in 320 may be utilized by the blood vessel detection unit 330 to accurately identify voxels in the real-time 3D image that correspond to blood vessels. With the identified blood vessel voxels, the model-based vessel centerline constructor 340 is provided to generate the centerline representation of the blood vessel structures. In this process, the 3D models for blood vessels in 320 may be utilized to extract the accurate vessel centerline representations 350 for blood vessel structures. The supplying vessel branch determine 360 is provided to select, from the detected blood vessels in accordance with supply condition specified in configuration 370, the vessel branches that may be used as supplying blood vessel branches for injecting the microsphere to deliver radioactive material with respect to the target region (tumor). The selection is performed based on the centerline representations for the detected blood vessels that can achieve a specified coverage of the target region with an as small a coverage as possible of the non-target region to minimize the damages to the healthy organs/tissues located in the non-target region. Details regarding how to leverage the centerline representations of blood vessels to identify supplying blood vessel branches is provided with reference to FIGS. 4A-6E.

Once the supplying blood vessel branches for delivering radioactive material to the target region, the branch injection point determiner 380 is provided to determine, on each of the supplying blood vessel branches, an injection point through which the microsphere with radioactive material is to be injected and delivered to some parts of the target region. Based on the supplying blood vessel branches (from the supplying vessel branch determiner 360) and the injection point (from the branch injection point determiner 380), the output 390 showing the supplying blood vessel branches with injection points thereon is generated and output. In some embodiments, the branch injection point determiner 380 may automatically determine an injection point for each supplying vessel branch. For instance, it may determine a point on the branch that corresponds to the largest diameter of the branch to make the injection safer. In some embodiments, the branch injection point determiner 380 may interact with a user, e.g., a surgeon or a nurse, to allow the user to hand pick an injection point based on, e.g., experience or the location of the vessel branch. In some embodiments, a semi-automated approach may be adopted by providing an automatically determined injection point to a user and allowing the user to modify via interactions.

FIG. 3B is a flowchart of an exemplary process of the framework 300 for automatically determining blood supplying vessel branches and injection points thereon with respect to a target region and a non-target region, in accordance with different embodiments of the present teaching. In operation, when real-time 3D images are received, voxels corresponding to blood vessels are identified at 305 therefrom. Such identified blood vessel voxels are then processed by the model-based vessel centerline constructor 340 to generate, at 315, vessel centerline representation 350 for different blood vessels. FIG. 3D illustrates an example vessel centerline representation 350 of a blood vessel tree, where center points of cross sections of a detected blood vessel structure are used to represent the detected blood vessel.

To select certain vessel branches for injecting radioactive material to reach a target region, the 3D model for the target region (i.e., 320-1) is retrieved, at 325, to determine the target region and non-target region in the real-time 3D images. With respect to such determine target region and non-target region in the organ, the supplying vessel branch determiner 360 selects, at 335, based on the conditions specified in 370 what qualifies as a supplying blood vessel, one or more supplying blood vessel branches as an optimal source to deliver radioactive material to the target region while minimize the damages to the non-target regions. The injection points on such supplying blood vessel branches are then determined, at 345, by the branch injection point determiner 380. The optimal supplying blood vessel branches and corresponding injection points thereon are then output at 355.

FIG. 4A depicts an exemplary system diagram of the supplying vessel branch determiner 360, in accordance with an embodiment of the present teaching. As discussed herein, the supplying vessel branch determiner 360 operates on the centerline representation of blood vessels detected from the real-time 3D images to identify, according to the supply condition specified in 370, the blood vessel branches that may be used to injecting microsphere to deliver radioactive material to the target region or a tumor. In some embodiments, the supplying vessel branches may be identified via operations of different stages. In the first stage, some centerline points on the centerline representation may be recognized as supplying centerline points according to some criterion. Each such recognized supplying point on the centerline representation represents a cross section of a blood vessel. In the second stage, adjacent supplying centerline points recognized in the first stage may be connected to form centerline segments representing candidate supplying vessel branches. In the third stage, an optimization operation is carried out to select some of the candidate supplying vessel branches in a manner so that when microsphere is injected therein, the radioactive material is delivered within the organ to reach or to cover the target region according to some specified criterion with a least coverage to the non-target region for the purpose of minimizing the damages to the healthy organ/tissues in the non-target region.

In this illustrated embodiment, the supplying vessel branch determiner 360 comprises a supplying centerline point determiner 400, a supplying centerline point filter 420, a supplying branch determiner 430, a supplying branch merge unit 440, and an optimal supplying branch generator 450. The supplying centerline point determiner 400 may be provided to perform the process of the first stage by assessing each centerline point in terms of whether it qualifies as a supplying point based on a supplying relation metric specified in 410. In some embodiments, the supplying relation metric may be defined via a distance measure such as the closest distance to assess how many locations in the organ (either in a target or a non-target region) that the centerline point can provide a supply. For example, if a centerline point is the closest centerline point to tissue point in the organ, then the centerline point is a supplying vessel point to the organ point.

FIG. 4B illustrates this concept via example vessel centerline points that are closest to tissue points in an organ, in accordance with an embodiment of the present teaching. In this illustration, a point in an organ 460 may be supplied with blood and radioactive material via the blood stream. A centerline point 470 may be identified as the closest vessel centerline point to 460 because the centerline point 470 is the closest to 460 with a distance dl. Given that, the centerline point 470 may correspond to a supplying centerline point to point 460. In most situations, the centerline point 470 may also be the supplying centerline point to tissues at other locations in the organ. Another similar example is centerline point 490, which is deemed as a supplying point to a tissue point 480 as it is closest to 480.

Each centerline point may be assessed based on its supplying ability measured by, e.g., the number of points in the target region it can supply radioactive material. To minimize the damage to normal tissues, it may also be recorded in terms of how many points the radioactive material may reach in non-target region(s). Such statistics may be collected in a process where the supplying centerline point for each of the points in the organ is determined according to which centerline point is in a closest distance. FIG. 4C illustrates example statistics recorded with respect to each centerline point, in accordance with an embodiment of the present teaching. In this example, the left column is a centerline point corresponding to a coordinate (X, Y, Z) of a voxel at the center of a cross section of a blood vessel in the real-time 3D image. For each centerline point, there are two recorded statistics, one L representing the total number of voxels the in the target region (e.g., within a lesion) that the centerline point can reach (i.e., closest to these lesion voxels) and the other N representing the total number of voxels in the non-target region (e.g., a normal tissue point) that the centerline point can reach. Such statistics are obtained by enumerate all voxels in a relevant region (e.g., the tumor and some surrounding region or the entire organ) in determining the closest centerline points.

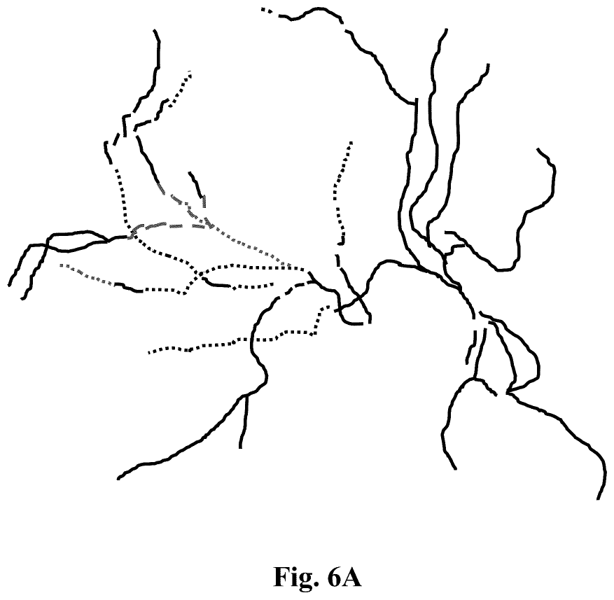

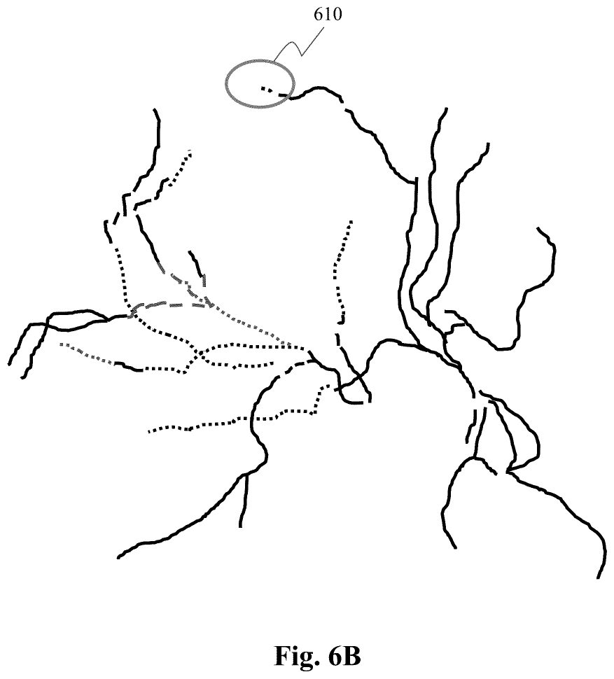

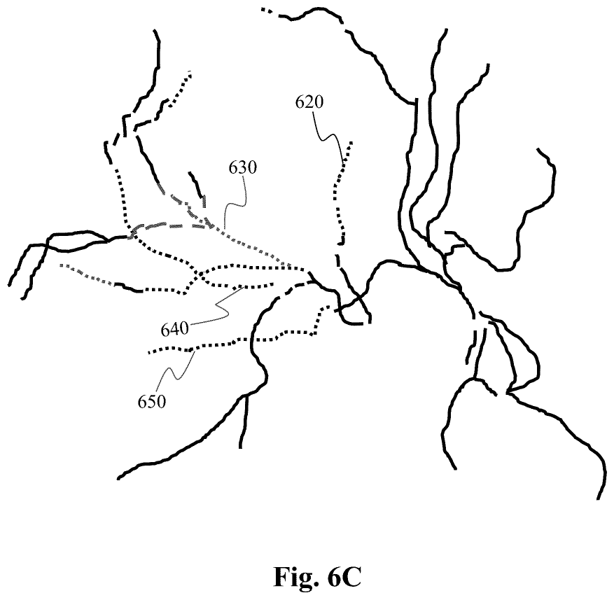

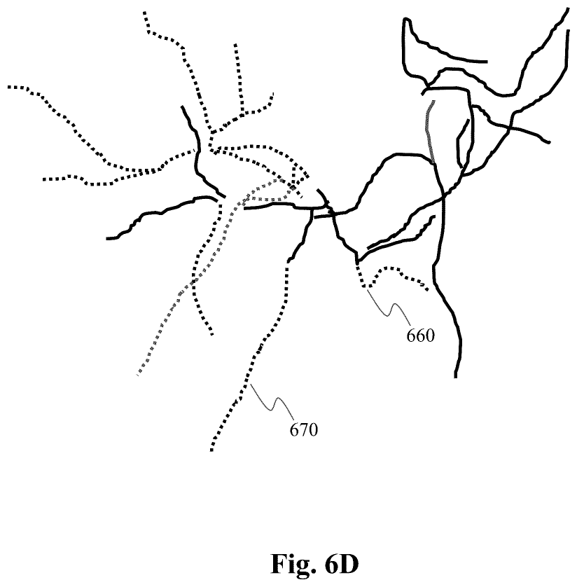

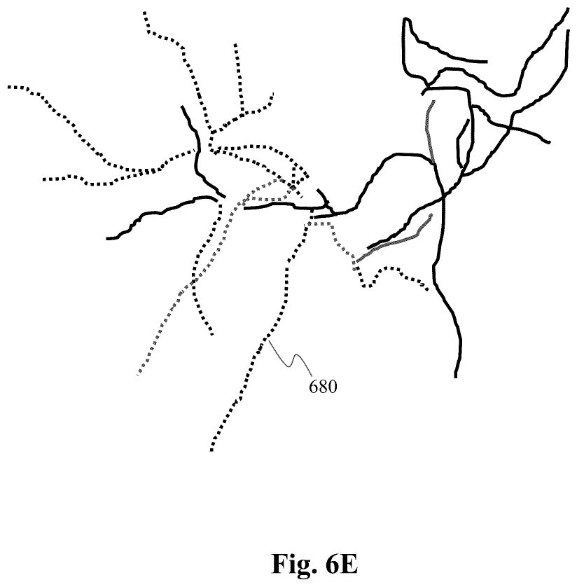

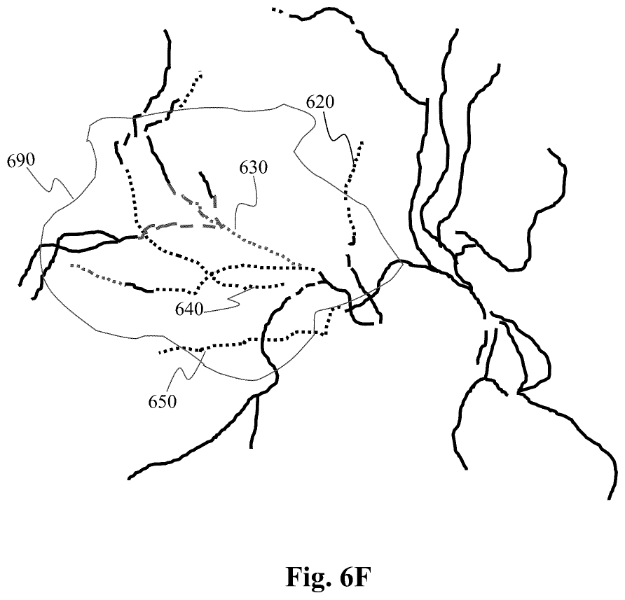

These statistics may be relied on to make several determinations. First, if a centerline point does not reach any target point or reaches only a few target points, the centerline point may be filtered out. The supplying centerline point filter 420 may be provided to perform the filtering in accordance with the condition to be satisfied specified in 370. For instance, any centerline point that has a target reason of fewer than a certain number (e.g., L<Lt) may be filtered out. In addition, any centerline point that can damage more non-target points than deliver treatment to target points (i.e., L<N) by a certain level (e.g., N−L>D) may be filtered out. The reaches associated with each centerline point may also be specifically recorded (not shown) with specific voxel coordinates that may be reached in both the target and non-target regions. FIG. 6A illustrates an example centerline representation with some points in green identified as supplying centerline vessels to the tumor region. FIG. 6B shows that some supplying centerline points in 610 are filtered out because, e.g., they supply to too few target points, or the non-target points they reach surpassed the number of target points.

The remaining supplying centerline points after the filtering may be used to form segments of centerline representation for supplying vessel branches. The supplying branch identifier 430 is provided to trace supplying centerline points to form centerline segments representing candidate supplying vessel branches. FIG. 6C shows such centerline segments formed based on the filtered result in FIG. 6B. As illustrated, by tracing the connected remaining supplying centerline points, 4 centerline segments are identified, labeled as 620, 630, 640, and 650, respectively. The statistics associated with each of the connected centerline points in the same centerline segment may be summed or consolidated to produce the statistics of the segment. As the target region 310-1 is detected based on the real-time 3D image, the coverage of the target region by each of the candidate supplying vessel branches (represented by the centerline segment) may be determined accordingly using the statistic on the number of voxels in the target region that the branch can reach. Similarly, the damage to the non-target region (which is outside of the target region) may also be determined based on the number of voxels in the non-target region that the radioactive material may reach from the branch.

In some embodiments, the centerline segments representing candidate supplying vessel branches may be merged in some situations to further consolidate. One reason for doing that may be that the number of branches that may be used for injecting microsphere may be minimized so that the injection point(s) for delivering the treatment may be minimized. The supplying branch merge unit 440 may be provided to do that. For example, any two centerline segments that can trace to a common centerline point may be merged into a larger centerline segment. FIG. 6D shows two centerline segments 660 and 670. If they are individually used as supplying vessel branches, each would need an injection point so that there are two injection points needed. As these two centerline segments share a common parent centerline point so that they may be merged into one centerline segment 680, as shown in FIG. 6E. To use the merged supplying vessel branch to deliver radioactive material, only one injection point may be needed. In some situations, two mergeable branches according to topology may not be merged. For instance, the merged supplying vessel branch may cause more damage than a specified level tolerance to normal tissue as a result of the merge. In this case, a merge may not be performed.

With the candidate supplying vessel branches identified at this stage of the processing, the optimal supplying branch generator 450 is provided for selecting some of the candidate supplying vessel branches to satisfy some specified criterion. Such criteria may be defined by a doctor according to, e.g., some desired treatment objective. For instance, if the procedure is applied to treat a malignant tumor in a target region, the treating doctor may specify that the injected microsphere with the radioactive material needs to reach at least 90% of the tumor. With this criterion, the optimization is to select a set of supplying vessel branches that can not only deliver the radioactive materials to the specified percent of tumor (coverage) with a least coverage to the non-target region to minimizing the damage of the radioactive material to the normal healthy tissues located in the non-target region.

FIG. 6F illustrates an example situation where, with respect to a target region 690 (e.g., a tumor that needs to be treated via selective internal radiation therapy), four supplying vessel branches identified are identified, including 620, 630, 640, and 650, each of which may reach a part of tumor 690 and may also impact negatively some healthy tissues outside of 690. Based on specific assessment on the coverages of both tumor region and non-tumor region of each supplying vessel branch and/or the coverages of different combinations of the identified supplying vessel branches, some may be retained as supplying vessel branches and some may not be selected as such when they are not necessarily needed to cover the target region yet unnecessarily cover non-target regions (e.g., normal tissue around a tumor) in order to minimize the damage to healthy tissues. For instance, branches 620 and 650 may not be selected as supplying vessel branches even though they each can also reach some tumor cells. As can be seen, each of these two branches, once injected with microsphere, the radioactive material likely will also unnecessarily reach more healthy tissues when compared with branches 630 and 640.

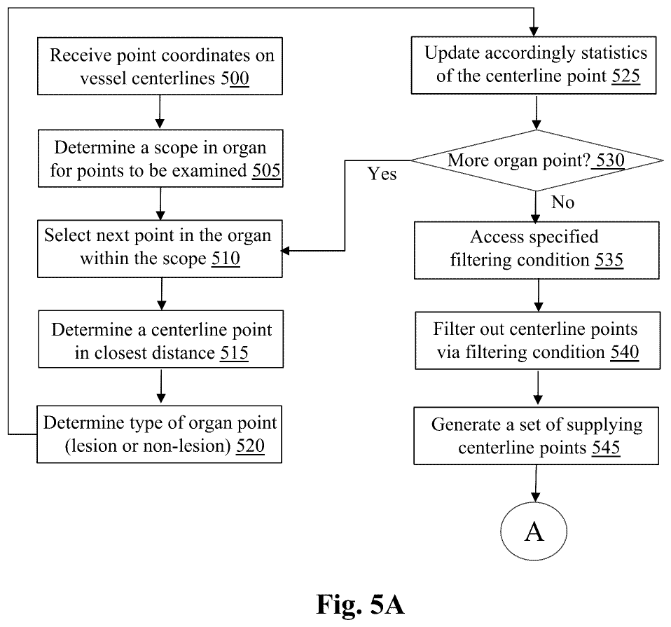

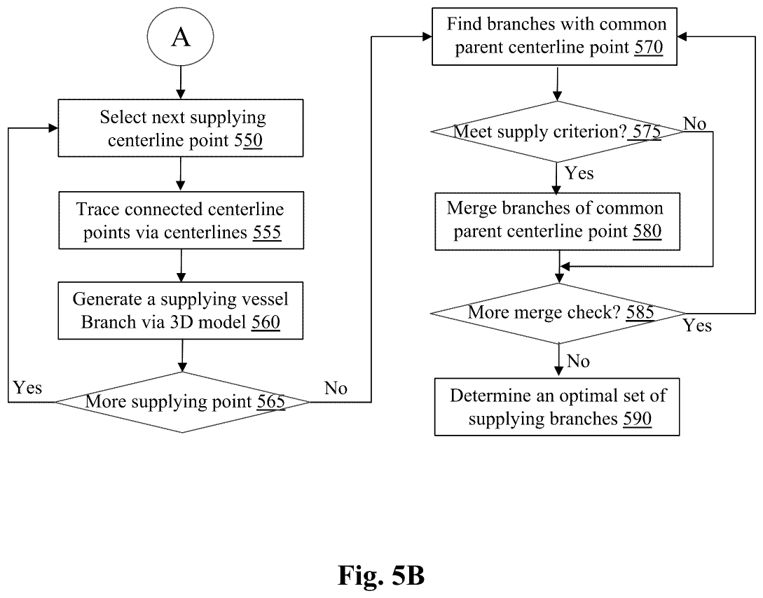

FIG. 5A is a flowchart of an exemplary process of the supplying vessel branch determiner 360 to identify supplying centerline points, in accordance with an embodiment of the present teaching. When the coordinates of voxels on the centerline representation of blood vessels are received at 500, the scope of organ points to be examined may be determined at 505. As discussed herein, the scope may be the entire real-time 3D image (which may include both the organ and nearby tissues), the entire organ region (e.g., detected from the real-time 3D image), or an area in the organ around a target region or a tumor. This scope may be used to assess relevant voxels in real-time 3D image as to whether they can be reached by a vessel to deliver radioactive material in a selective internal radiation therapy. In operation, for each voxel within the scope chosen at 510, a point on the centerline representation for blood vessels with the closest distance to the chosen voxel may be determined at 515. Based on the nature of the voxel (in the target or the non-target region), determined at 520, the statistics associated with the centerline point may be accordingly updated at 525. The process continues until, determined at 530, all voxels in the scope are processed. As discussed herein, FIG. 6A shows an example result of this process, where the centerline points in green represent the ones that can reach some tumor voxels.

To filter out centerline points that may reach only a negligible number of tumor cells, the supplying centerline point filter 420 accesses, at 535, specified supply condition 370 and filters, at 540, the identified supplying centerline points accordingly to generate, at 545, a set of supplying centerline points that satisfy the supply condition 370. FIG. 6B shows some centerline points in 610 that are filtered out during this step.

FIG. 5B is a flowchart of an exemplary continued process of the supply vessel branch determiner 360 to generate an optimal set of supplying vessel branches with respect to a target region, in accordance with an embodiment of the present teaching. With the set of supplying centerline points from step 545, the supplying branch identifier 430 traces each of the supplying centerline points to their adjacent supplying centerline points until all supplying centerline points are processed. In an iterative process, for a next supplying centerline selected at 550, supplying centerline points connected thereto are traced, at 555, to generate a supplying centerline segment, as discussed herein. This is illustrated in FIG. 6C with supplying vessel segments 620-650. Each such identified supplying centerline segment may then be used to generate, at 560, a supplying vessel branch. FIG. 2B illustrates some supplying vessel branches. In some embodiments, transforming a supplying centerline segment to a supplying vessel branch may be achieved based on 3D models representing blood vessels. The process of generating supplying vessel branches continues until, determined at 565, all supplying centerline points are considered.

As discussed herein, in some embodiments, further optimization may be performed by merging different supplying vessel branches. The supplying branch merge unit 440 identifies, at 570, supplying centerline segments (representing supplying vessel branches) that may trace to a common parent centerline point in the centerline representation. In certain situations, such identified vessel branches may be merged if the merge satisfies certain criterion (e.g., the merge will not cause a certain level of added damage to healthy tissues). This is determined at 575. If so, the two supplying vessel branches may then be merged at 580. Otherwise, no merge is carried out. The merge process continues until, determined at 585, all pairs of merge candidates are considered. At that point, based on the currently identified supplying vessel branches, the optimal supplying branch generator 450 determines, at 590, an optimal set of supplying vessel branches identified according to the present teaching that can maximally cover the target region yet minimize the damages to healthy tissues in the non-target region, as illustrated in FIG. 6F.

As discussed herein, for each centerline point in each of the supplying centerline segments, statistics are collected for each point directed to its coverage to both the target voxels (e.g., the number of voxels in the target region that the centerline point can reach) and the non-target region (e.g., the number of voxels in the non-target region that the centerline point can reach). This is illustrated in FIG. 4C. To select optimal supplying vessel branches, with respect to each supplying vessel branch, similar statistics may be estimated to indicate the coverage of the supplying branch to both target and non-target voxels. In some embodiments, this may be achieved by cumulating corresponding statistics associated with all centerline points along the supplying centerline segment representing a supplying vessel branch. Such cumulated statistics represent the overall coverage of the supplying vessel branch to both target and non-target voxels. With such statistics available for each supplying vessel branch, a set of optimal supplying vessel branches may be selected so that, in combination, the selected supplying vessel branches together can satisfy a desired coverage over the target-region and a least coverage over the non-target region to ensure a minimum damage to healthy tissues in the non-target region. In practice, the level of coverage for the target-region may be determined by the physician based on the goal of the treatment to the underlying illness. Similarly, the level of damage allowed may also be specified in each application based on the location of the illness and physiology around the region to be treated.

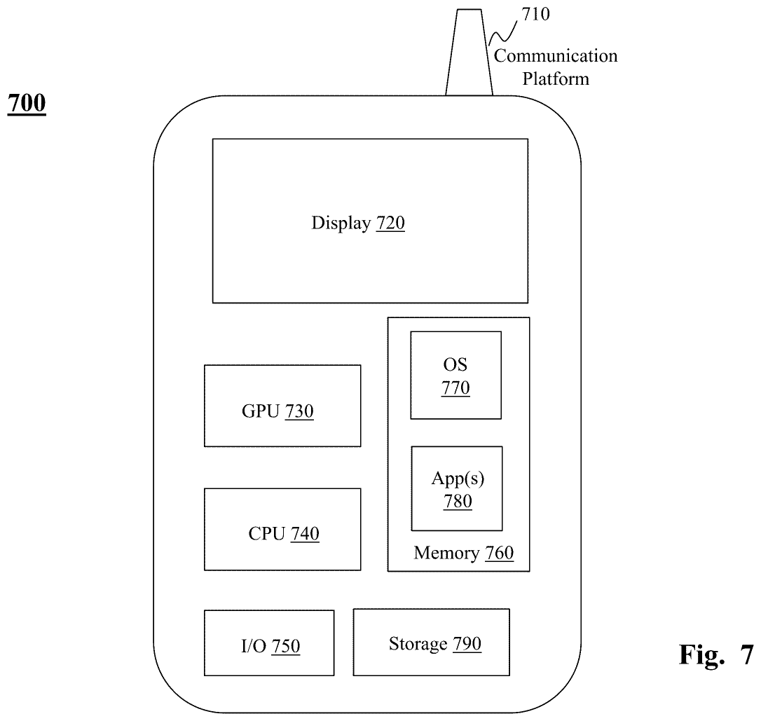

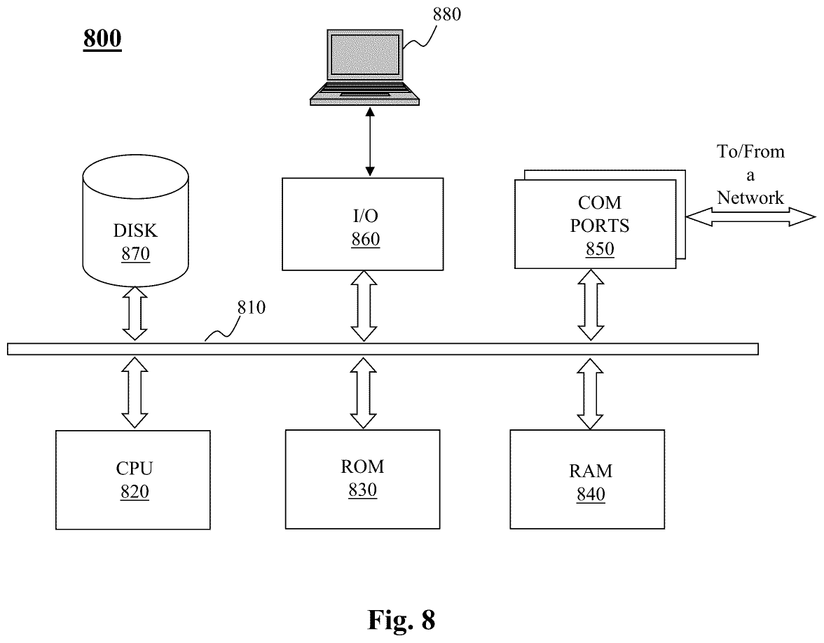

FIG. 7 is an illustrative diagram of an exemplary mobile device architecture that may be used to realize a specialized system implementing the present teaching in accordance with various embodiments. In this example, the user device on which the present teaching may be implemented corresponds to a mobile device 700, including, but not limited to, a smart phone, a tablet, a music player, a handled gaming console, a global positioning system (GPS) receiver, and a wearable computing device, or in any other form factor. Mobile device 700 may include one or more central processing units (“CPUs”) 740, one or more graphic processing units (“GPUs”) 730, a display 720, a memory 760, a communication platform 710, such as a wireless communication module, storage 790, and one or more input/output (I/O) devices 750. Any other suitable component, including but not limited to a system bus or a controller (not shown), may also be included in the mobile device 700. As shown in FIG. 8, a mobile operating system 770 (e.g., iOS, Android, Windows Phone, etc.), and one or more applications 780 may be loaded into memory 760 from storage 790 in order to be executed by the CPU 740. The applications 780 may include a user interface or any other suitable mobile apps for information analytics and management according to the present teaching on, at least partially, the mobile device 700. User interactions, if any, may be achieved via the I/O devices 750 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

FIG. 8 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 800 may be used to implement any component or aspect of the framework as disclosed herein. For example, the information analytical and management method and system as disclosed herein may be implemented on a computer such as computer 800, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 800, for example, includes COM ports 850 connected to and from a network connected thereto to facilitate data communications. Computer 800 also includes a central processing unit (CPU) 820, in the form of one or more processors, for executing program instructions. The exemplary computer platform includes an internal communication bus 810, program storage and data storage of different forms (e.g., disk 870, read only memory (ROM) 830, or random-access memory (RAM) 840), for various data files to be processed and/or communicated by computer 800, as well as possibly program instructions to be executed by CPU 820. Computer 800 also includes an I/O component 860, supporting input/output flows between the computer and other components therein such as user interface elements 880. Computer 800 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method, comprising:
receiving a three-dimensional (3D) image with voxels capturing an organ with target and non-target regions and one or more blood vessels therein;
generating a centerline representation with a plurality of centerline points representing the one or more blood vessels;
identifying, from the centerline representation, at least one supplying centerline segment, each of which includes multiple connected supplying centerline points and represents a supplying vessel among the one or more blood vessels; and
selecting an optimal set of supplying vessels that satisfy a specified coverage to the target region with a least coverage to the non-target region.

2. The method of claim 1, wherein each of the plurality of centerline points in the centerline representation corresponds to a center point of a cross section of the one or more blood vessels.

3. The method of claim 1, wherein the step of identifying the at least one supplying centerline segment comprises:
with respect to each voxel in the 3D image in both the target and the non-target regions,
identifying a centerline point in the centerline representation having a closest distance to the voxel,
incrementing a first statistic associated with the centerline point indicative of a coverage of the centerline point for the target region if the voxel is in the target region, and
incrementing a second statistic associated with the centerline point indicative of a coverage of the centerline point for the non-target region if the voxel is in the non-target region.

4. The method of claim 3, further comprising:
with respect to each of the plurality of centerline points with associated first and second statistics,
determining whether the centerline point covers a minimum number of voxels in the target region,
classifying, if the centerline point covers a minimum number of voxels in the target region, the centerline point as a supplying centerline point; and
connecting supplying centerline points that are adjacent in the centerline representation to form a candidate supplying centerline segment.

5. The method of claim 4, further comprising:
with respect to each candidate supplying centerline segment,
determining whether the candidate supplying centerline segment satisfies a pre-determined condition,
retaining the candidate supplying centerline segment if the pre-determined condition is satisfied, and
discarding the candidate supplying centerline segment if the pre-determined condition is not satisfied.

6. The method of claim 5, further comprising merging some of the retained candidate supplying centerline segments that have a common parent centerline point to generate an updated candidate supplying centerline segment.

7. The method of claim 1, wherein the step of selecting an optimal set of supplying vessel branches comprises:
estimating, with respect to each of the at least one supplying centerline segment, respective coverages to the target and non-target regions based on corresponding statistics associated with each of the supplying centerline points in the supplying centerline segment; and
for each combination of the at least one supplying centerline segment, determining:
a target coverage to the target region based on corresponding coverages to the target region related to individual supplying centerline segments in in the combination,
a non-target coverage to the non-target region based on corresponding coverages to the non-target region of the individual supplying centerline segments;
selecting one of the combinations as the set of optimal supplying vessel branches that:
satisfies the specified coverage to the target region, and
has the least coverage to the non-target region.

8. The method of claim 7, wherein
the specified coverage to the target region indicates a first percent of voxels in the target region in the 3D image; and
the coverage to the non-target region indicates a second percent of voxels in the non-target region in the 3D image.

9. The method of claim 1, further comprising determining, with respect to each supplying vessel branches represented by each corresponding supplying centerline segment in the optimal set, an injection point for injecting microsphere with radioactive material to deliver radioactive material to the target region in a selective internal radiation therapy.

10. A system, comprising:
a model-based vessel centerline constructor implemented by a processor and configured for:
receiving a three-dimensional (3D) image with voxels capturing an organ with target and non-target regions and one or more blood vessels therein,
generating a centerline representation with a plurality of centerline points representing the one or more blood vessels; and
a supplying vessel branch determiner implemented by a processor and configured for:
identifying, from the centerline representation, at least one supplying centerline segment, each of which includes multiple connected supplying centerline points and represents a supplying vessel among the one or more blood vessels, and
selecting an optimal set of supplying vessels that satisfy a specified coverage to the target region with a least coverage to the non-target region.

11. The system of claim 10, wherein each of the plurality of centerline points in the centerline representation corresponds to a center point of a cross section of the one or more blood vessels.

12. The system of claim 10, wherein the step of identifying the at least one supplying centerline segment comprises:
with respect to each voxel in the 3D image in both the target and the non-target regions,
identifying a centerline point in the centerline representation having a closest distance to the voxel,
incrementing a first statistic associated with the centerline point indicative of a coverage of the centerline point for the target region if the voxel is in the target region, and
incrementing a second statistic associated with the centerline point indicative of a coverage of the centerline point for the non-target region if the voxel is in the non-target region.

13. The system of claim 12, further comprising:
with respect to each of the plurality of centerline points with associated first and second statistics,
determining whether the centerline point covers a minimum number of voxels in the target region,
classifying, if the centerline point covers a minimum number of voxels in the target region, the centerline point as a supplying centerline point; and
connecting supplying centerline points that are adjacent in the centerline representation to form a candidate supplying centerline segment.

14. The system of claim 13, further comprising:
with respect to each candidate supplying centerline segment,
determining whether the candidate supplying centerline segment satisfies a pre-determined condition,
retaining the candidate supplying centerline segment if the pre-determined condition is satisfied, and
discarding the candidate supplying centerline segment if the pre-determined condition is not satisfied.

15. The system of claim 14, further comprising merging some of the retained candidate supplying centerline segments that have a common parent centerline point to generate an updated candidate supplying centerline segment.

16. The system of claim 10, wherein the step of selecting an optimal set of supplying vessel branches comprises:
estimating, with respect to each of the at least one supplying centerline segment, respective coverages to the target and non-target regions based on corresponding statistics associated with each of the supplying centerline points in the supplying centerline segment; and
for each combination of the at least one supplying centerline segment, determining:
a target coverage to the target region based on corresponding coverages to the target region related to individual supplying centerline segments in in the combination,
a non-target coverage to the non-target region based on corresponding coverages to the non-target region of the individual supplying centerline segments;
selecting one of the combinations as the set of optimal supplying vessel branches that:
satisfies the specified coverage to the target region, and
has the least coverage to the non-target region.

17. The system of claim 16, wherein
the specified coverage to the target region indicates a first percent of voxels in the target region in the 3D image; and
the coverage to the non-target region indicates a second percent of voxels in the non-target region in the 3D image.

18. The system of claim 10, further comprising a branch injection point determiner implemented by a processor and configured for determining, with respect to each supplying vessel branches represented by each corresponding supplying centerline segment in the optimal set, an injection point for injecting microsphere with radioactive material to deliver radioactive material to the target region in a selective internal radiation therapy.

19. A machine-readable and non-transitory medium having information recorded thereon, wherein the information, when read by the machine, causes the machine to perform the following steps:
receiving a three-dimensional (3D) image with voxels capturing an organ with target and non-target regions and one or more blood vessels therein;
generating a centerline representation with a plurality of centerline points representing the one or more blood vessels;
identifying, from the centerline representation, at least one supplying centerline segment, each of which includes multiple connected supplying centerline points and represents a supplying vessel among the one or more blood vessels; and
selecting an optimal set of supplying vessels that satisfy a specified coverage to the target region with a least coverage to the non-target region.

20. The medium of claim 19, wherein the step of identifying the at least one supplying centerline segment comprises:
with respect to each voxel in the 3D image in both the target and the non-target regions,
identifying a centerline point in the centerline representation having a closest distance to the voxel,
incrementing a first statistic associated with the centerline point indicative of a coverage of the centerline point for the target region if the voxel is in the target region, and
incrementing a second statistic associated with the centerline point indicative of a coverage of the centerline point for the non-target region if the voxel is in the non-target region.

21. The medium of claim 20, wherein the information, when read by the machine, further causes the machine to perform the following steps:
with respect to each of the plurality of centerline points with associated first and second statistics,
determining whether the centerline point covers a minimum number of voxels in the target region,
classifying, if the centerline point covers a minimum number of voxels in the target region, the centerline point as a supplying centerline point; and
connecting supplying centerline points that are adjacent in the centerline representation to form a candidate supplying centerline segment.

22. The medium of claim 21, wherein the information, when read by the machine, further causes the machine to perform the following steps:
with respect to each candidate supplying centerline segment,
determining whether the candidate supplying centerline segment satisfies a pre-determined condition,
retaining the candidate supplying centerline segment if the pre-determined condition is satisfied, and
discarding the candidate supplying centerline segment if the pre-determined condition is not satisfied.

23. The medium of claim 22, wherein the information, when read by the machine, further causes the machine to perform the step of merging some of the retained candidate supplying centerline segments that have a common parent centerline point to generate an updated candidate supplying centerline segment.

24. The medium of claim 19, wherein the step of selecting an optimal set of supplying vessel branches comprises:
estimating, with respect to each of the at least one supplying centerline segment, respective coverages to the target and non-target regions based on corresponding statistics associated with each of the supplying centerline points in the supplying centerline segment; and
for each combination of the at least one supplying centerline segment, determining:
a target coverage to the target region based on corresponding coverages to the target region related to individual supplying centerline segments in in the combination,
a non-target coverage to the non-target region based on corresponding coverages to the non-target region of the individual supplying centerline segments;
selecting one of the combinations as the set of optimal supplying vessel branches that:
satisfies the specified coverage to the target region, and
has the least coverage to the non-target region.

25. The medium of claim 24, wherein
the specified coverage to the target region indicates a first percent of voxels in the target region in the 3D image; and
the coverage to the non-target region indicates a second percent of voxels in the non-target region in the 3D image.

26. The medium of claim 19, wherein the information, when read by the machine, further causes the machine to perform the step of determining, with respect to each supplying vessel branches represented by each corresponding supplying centerline segment in the optimal set, an injection point for injecting microsphere with radioactive material to deliver radioactive material to the target region in a selective internal radiation therapy.
